(19) United States
(12) Patent Application Publication
(10) Pub. No.: US 2006/0175556 A1
(43) Pub. Date: Aug. 10, 2006

(54) ILLUMINATION OPTICAL SYSTEM, EXPOSURE APPARATUS, AND DEVICE MANUFACTURING METHOD

(75) Inventor: AKIRA YABUKI, Shimotsuga-gun (JP)

Correspondence Address: FITZPATRICK CELLA HARPER & SCINTO, 30 ROCKEFELLER PLAZA, NEW YORK, NY, 10112, US

(73) Assignee: CANON KABUSHIKI KAISHA, Tokyo (JP)

(21) Appl. No.: 11/275,957

(22) Filed: Feb. 7, 2006

(30) Foreign Application Priority Data
Feb. 7, 2005 (JP) 2005-031082

(51) Int. Cl. G21G 5/00; A61N 5/00

(52) U.S. Cl. 250/492.1

(57) ABSTRACT

An illumination optical system for illuminating a target plane by using light from a light source includes plural displaceable mirrors that are two-dimensionally arranged at specific positions in said illumination optical system.

### BACKGROUND OF THE INVENTION

The present invention generally relates to an illumination optical system, and more particularly to an illumination optical system, an exposure apparatus and a device manufacturing method, which use extreme ultraviolet (“EUV”) light having a wavelength between 5 nm and 20 nm to expose a substrate, such as a single crystal substrate for a semiconductor wafer, and a glass plate for a liquid display device (“LCD”).

The conventional illumination optical system in a semiconductor exposure apparatus enables an optical element, such as a lens, to be moved along an optical-axis direction in changing an illumination condition, such as a coherence factor σ (a ratio between the numerical aperture (“NA”) of the illumination optical system at the mask side and the NA of the projection optical system at the mask side) in a normal illumination, and a shape of a off-axis illumination, e.g., an annular ratio in the annular illumination (a ratio between an internal σ and an external σ).

In the semiconductor exposure apparatus that uses an EUV light source, each optical element shows a small reflectance. Therefore, for the off-axis illumination, it is known that a mechanical aperture stop, such as an iris stop, can more efficiently use the light than a system that combines plural optical elements.

One proposed illumination optical system has a turret mechanism that arranges plural aperture stops on the pupil plane of the illumination optical system so as to change the illumination condition, such as a coherence factor and a shape of the off-axis illumination. The illumination optical system enables one of the aperture stops to be selected in accordance with a desired illumination condition. See, for example, Japanese Patent Application, Publication No. 2002-203767 (paragraph no. 0081 and FIG. 4). Another proposed illumination optical system has, on the pupil plane, a reflective integrator that arranges cylindrical reflective surfaces in parallel or arranges fine reflective surfaces two-dimensionally. This illumination optical system enables the reflective integrator to be changed in accordance with the illumination condition. See, for example, Japanese Patent Application, Publication No. 2003-045784 (paragraph nos. 0041-0042, 0082, and FIGS. 2, 7, 12, etc.).

Still another example is an EUV exposure apparatus that uses a bundle of light sources to increase the light intensity, and arranges an orientation-variable mirror in the illumination optical system. See, for example, Japanese Patent Application, Publication No. 2003-185798 (paragraph no. 0012 and FIG. 4, etc.).

However, those configurations which arrange plural aperture stops on the turret and switch plural integrators provide only a few illumination conditions available, and thus cannot provide an arbitrary and continuous illumination condition. While the number of available illumination conditions can be increased by increasing the number of aperture stops and the number of integrators, this measure would enlarge the exposure apparatus and thus not be realistic.

Moreover, the iris stop and the turret switching mechanism for changing the illumination condition need a comparatively large actuator to be installed in a vacuum area that houses the illumination optical system to change the illumination condition. The large actuator has a large surface area, emits a large amount of gas, and destroys the degree of vacuum in the vacuum area.

Moreover, in exchanging the aperture stop mounted on the turret etc. from the outside of the vacuum area, the vacuum purge is destroyed once and the atmosphere is opened to the air before the exchange operation starts. This procedure extremely lowers the operating efficiency of the exposure apparatus.

### BRIEF SUMMARY OF THE INVENTION

The present invention is directed to an illumination optical system, exposure apparatus and device manufacturing method, which arbitrarily and continuously change the illumination condition.

An illumination optical system according to one aspect of the present invention for illuminating a target plane by using light from a light source includes plural displaceable mirrors that are two-dimensionally arranged at specific positions in the illumination optical system.

An exposure apparatus according to another aspect of the present invention includes the illumination optical system for illuminating an original, and a projection optical system for projecting an image of a pattern of the original onto a substrate.

A device manufacturing method according to still another aspect of the present invention includes the steps of exposing a substrate using the above exposure apparatus, and developing the substrate that has been exposed.

Other objects and further features of the present invention will become readily apparent from the following description of the preferred embodiments with reference to the accompanying drawings.

### BRIEF DESCRIPTION OF THE DRAWINGS

FIG. 1 is a schematic structural view of an exposure apparatus having an illumination optical system according to a first embodiment of the present invention.

FIGS. 2A to 2C are views for explaining an integrator provided in the illumination optical system according to the first embodiment.

FIG. 3 is a view for explaining a sectional shape and an annular illumination of the integrator.

FIG. 4 is a structural view of one fine mirror in the integrator.

FIG. 5 is a view of an integrator according to another embodiment.

FIG. 6 is a flowchart showing a mirror driving control of the integrator.

FIG. 7 is a flowchart for explaining a device manufacturing method using the exposure apparatus according to the first embodiment.

FIG. 8 is a flowchart for explaining a device manufacturing method using the exposure apparatus according to the first embodiment.

### DETAILED DESCRIPTION OF THE PREFERRED EMBODIMENTS

### First Embodiment

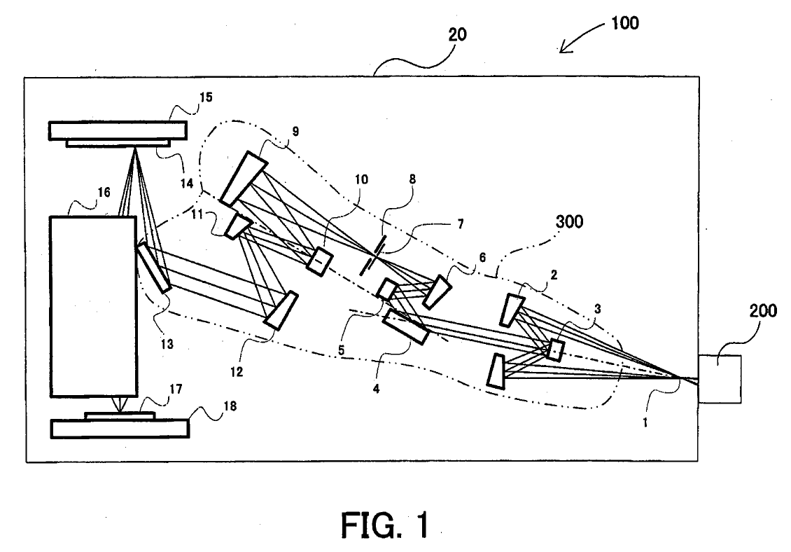

FIG. 1 shows a schematic structure of a semiconductor exposure apparatus that includes an illumination optical system according to a first embodiment of the present invention. The exposure apparatus 100 of this embodiment is a projection exposure apparatus that provides step-and-scan exposure using the EUV light that has, for example, a wavelength of 13.4 nm, as an illumination light for exposure.

The exposure apparatus includes a light source section 200, an illumination optical system 300, a reflection type reduction projection optical system 16, a mask stage 15 that holds a reflection mask (or reticle) 14 as an original, and a wafer stage 18 that holds a semiconductor wafer 17 as a substrate to be exposed. The mask stage 15 and the wafer stage 18 are connected to a controller (not shown) so that the controller can control their driving. The mask stage 15 positions the mask 14, and the wafer stage 18 positions the wafer 17. The light source section 200 and the illumination optical system 300 constitute an illumination apparatus. While this embodiment discusses the exposure apparatus that uses the reflection mask, the present invention is applicable to another type of mask.

The EUV light has low transmittance to the air, and the light source section 200 is housed in the vacuum chamber. Other components, such as the illumination optical system 300 and the projection optical system 16, are also housed in the vacuum chamber 20. The illumination optical system 300 illuminates the mask 14 uniformly using the arc-shaped EUV light corresponding to the arc-shaped field of the reflection type reduction projection optical system 16.

In the illumination optical system 300, 1 denotes a light source image that is formed by condensing, through a mirror (not shown), the EUV light emitted from a plasma light emitting point in the light source section 200. 2 and 3 form a collimating (or first) optical system that includes concave and convex mirrors, and converts the EUV light from the light source image 1 into an approximately parallel light.

4 denotes an integrator having plural fine cylindrical surface mirrors, which will be described in detail. The integrator 4 is arranged on or near the pupil plane of the illumination optical system 200.

5 and 6 form an optical system that includes a parabolic mirror that condenses the light from the integrator 4 in an arc shape. The integrator 4 and optical system 5, 6 form an arcing optical system.

7 denotes a slit having an arc opening. 8 denotes a masking blade that restricts the illumination light to a desired exposure area. The masking blade 8 includes an opening that passes the EUV light, and the light-shielding part that is made of a material that absorbs the EUV light, and shields the unnecessary stray light that does not contribute to the arc illumination. The slit 7 together with a slit-width adjusting mechanism (not shown) sets a desired slit width and partially changes the slit width, thereby successfully correcting the uneven light intensity.

9, 10, 11 and 12 denote curved mirrors that form a mask imaging system. 13 denotes a plane mirror that reflects the image-side light of the mask imaging system 9-12 toward the oblique upper side, and introduces the light to the mask 14 held on the mask stage 15 at a predetermined angle.

The arc-shaped EUV light that has passed the slit 7 and the masking blade 8 is converted by the mask imaging system 9 into a desired magnification, is deflected on the plane mirror 13, and forms the arc-shaped illumination area on the mask 14. The arcing optical system 5-6, the slit 7, the masking blade 8, and the masking blade 8, and the mask imaging system 9-12 form a second optical system.

The reflection type reduction projection optical system 16 includes plural mirror (not shown), and projects an image of the light onto the wafer 17 on the wafer stage 18, which light is reflected light from the mask 14, and contains pattern information formed on the mask 14.

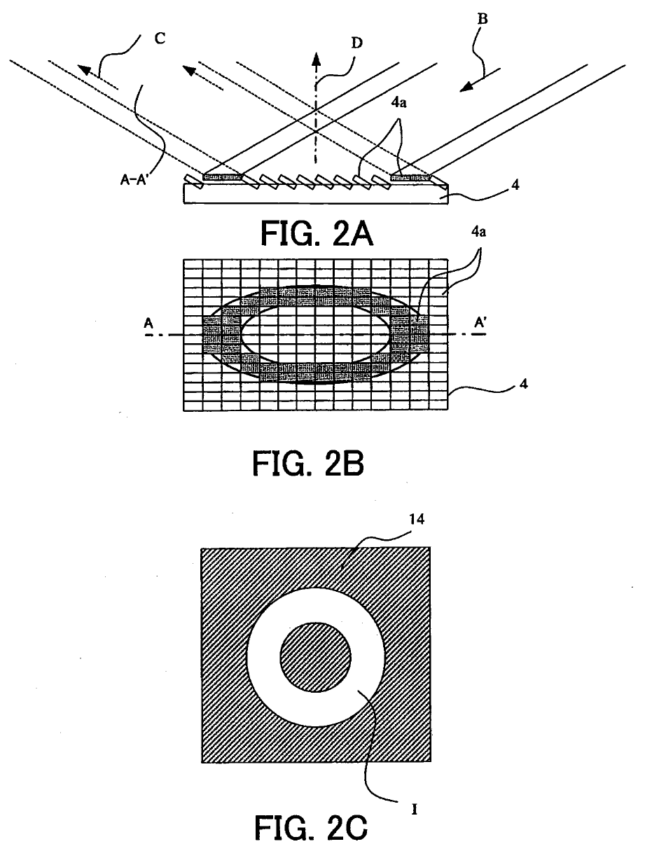

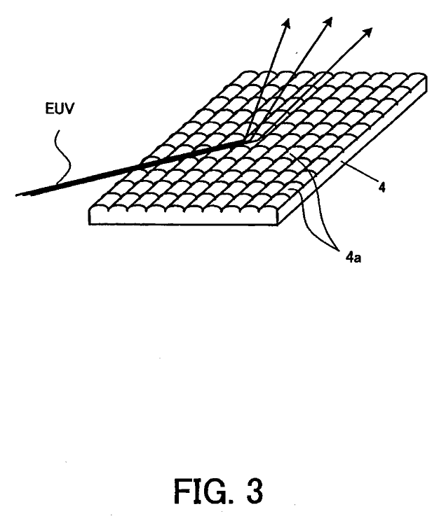

Referring to FIGS. 2 to 4, a description will be given of the integrator 4. FIG. 3 is a detailed view of the integrator 4.

As shown in FIG. 3, the integrator 4 has plural, two-dimensionally arranged at a repetitive period, fine convex cylindrical surface mirrors 4a. These cylindrical surface mirrors 4a are arranged so that their generating lines are parallel with each other.

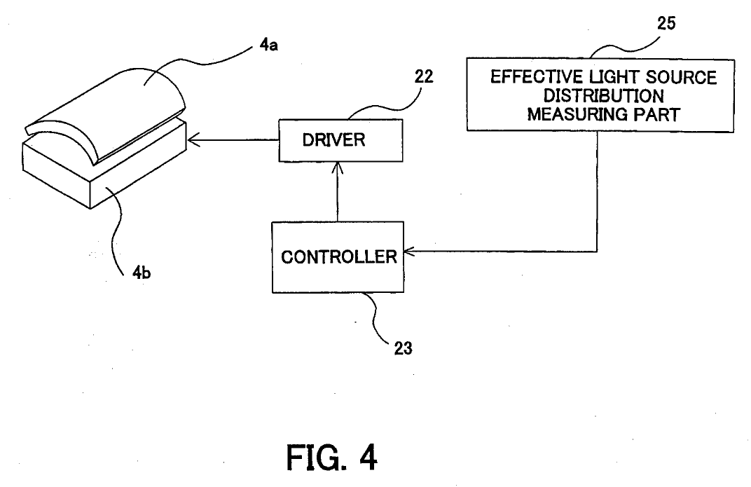

In addition, as shown in FIG. 4, each cylindrical surface mirror 4a is provided with an actuator 4b. The cylindrical surface mirror 4a is driven and displaced as the actuator 4b is operated. Thereby, the orientation of the mirror 4a, i.e., at least one of an inclination and a position (or a height in the light incident/reflecting direction), is variable continuously or smoothly. This embodiment provides one actuator for one cylindrical surface mirror 4a, and allows all the cylindrical surface mirrors 4a to be varied independently.

The actuator 4b may be a layered or bimorph type piezoelectric actuator, an electromagnetic coil actuator, or an inchwarm actuator. The mirror 4a may be displaced by unidirectional, bidirectional, or multi-directional driving.

The integrator reflects part or whole of the incident EUV light to the arcing optical system 5-6 by some or all of the mirrors 4a. When the parabolic mirror in the arcing optical system 5-6 condenses and superimposes the light, the arc-shaped illumination area has an approximately uniform light intensity distribution.

FIG. 3 schematically shows that almost parallel light incident upon the integrator 4 and reflected on it. The incident, approximately parallel light divergently proceeds due to the reflection on the convex cylindrical surface of the mirror 4a. In other words, when approximately parallel EUV light is incident upon the mirror 4a having the cylindrical surface, the secondary light source is formed at the reflecting position. The angular distribution of the EUV light emitted from the secondary light source has a conical shape. The secondary light source exists as a virtual image inside the convex cylindrical reflection surface. The arc-shaped illumination is made by reflecting the EUV light with the mirror having a focal point at the secondary light source position, and by illuminating the mask 14 or the surface conjugate with the mask 14.

Orientations of some or all of the mirrors 4a are controllable in the integrator 4. Thereby, the illumination condition is variable, such as a coherence factor σ in a normal illumination and a shape ratio (e.g., an annular ratio of an annular illumination) of a off-axis illumination, such as the annular illumination and a quadrupole illumination.

FIG. 2B is a top view of the integrator 4. FIG. 2A is a sectional view taken along line A-A′ in FIG. 2B. These figures show an orientation of each mirror when the mask 14 is illuminated by the annular illumination, as shown in FIG. 2C.

As shown in FIG. 2A, the EUV light is incident obliquely upon the integrator 4 along the arrow B direction from the collimating optical systems 2 and 3. The EUV light reflected by the mirror 4a highlighted in FIG. 2A (which is approximately parallel to the lateral direction in FIG. 2A) proceeds towards the arcing optical system 5-6 in the arrow C direction, forming the light intensity distribution (effective light source distribution) for the annular illumination. In the integrator 4, the plane of each mirror 4a (“reflecting surface” hereinafter) is conjugate with the pupil plane of the projection optical system 16. Therefore, the light intensity distribution on the reflecting surface of the integrator 4 corresponds to the light intensity distribution on the pupil plane of the projection optical system 16 or the effective light source distribution.

On the other hand, in FIG. 2A, among the EUV lights incident along the arrow B direction, the EUV light reflected by the mirror 4a other than the highlighted mirror 4a proceeds towards the arrow D direction. A light absorber (not shown) is provided ahead of the arrow D direction, and processes the light as heat energy.

Thus, a σ value of a normal illumination and an annular ratio in the annular illumination can be continuously changed by selecting the orientation-variable mirror 4a. Of course, a desired off-axis illumination is available, such as dipole, quadrupole, and sextupole illuminations.

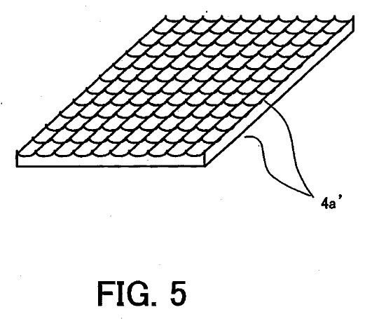

While FIG. 3 describes the integrator 4 that arranges the mirrors 4a each having a convex cylindrical surface, mirrors 4a each having a concave cylindrical surface may be arranged as shown in FIG. 5. In this case, the secondary light source exists as a virtual image outside the concave cylindrical reflecting surface, but the concave cylindrical surface provides the same effect as the convex cylindrical surface.

Furthermore, two types of convex and concave cylindrical surface mirrors 4a and 4a′ may be arranged in the same integrator 4.

As shown in FIG. 4, each actuator 4b is connected to a driver 22 that operates the actuator 4b, and each driver 22 is connected to a controller 23 that controls the displacement driving of each actuator 4b.

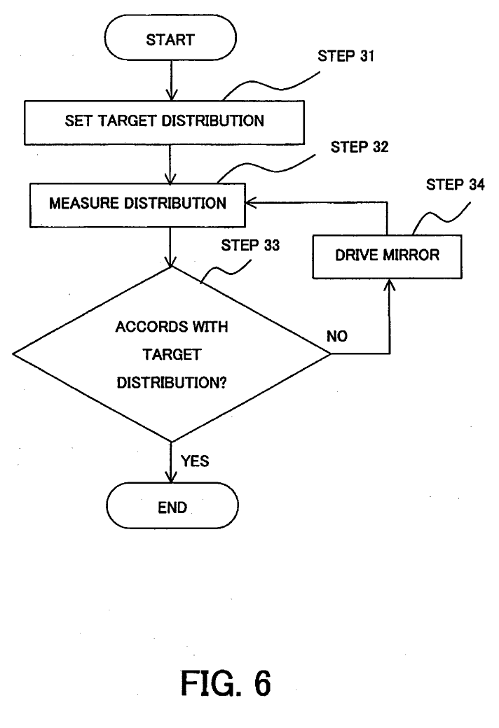

In FIG. 4, 25 denotes a measuring part that measures a light intensity distribution corresponding to the effective light source distribution, which light intensity distribution is referred to as the “effective light source distribution” hereinafter. FIG. 6 is a flowchart for explaining the illumination condition control operation of the controller 23.

The target effective light source distribution (“target distribution” hereinafter) is previously set or input to the controller 23 (step 31). The controller 23 instructs the measuring part 25 to measure the current effective light source distribution (step 32), and determines whether the measured effective light source distribution accords with the target distribution (step 33). If not, the procedure moves to step 34, which drives the mirror 4a in such a direction that the effective light source distribution approaches to the target distribution. On the other hand, if so, the procedure ends.

The feedback control of the orientation of the mirror 4a so that the measured effective light source distribution approaches to the target distribution renders the effective light source distribution optimal to the exposure pattern. As discussed, this embodiment can independently control the orientation of each mirror 4a in the integrator 4, and arbitrarily and continuously vary the illumination condition, such as a σ value of a normal illumination and an annular ratio in the annular illumination. This configuration thus can eliminate the large switching mechanism that uses the turret described in the background of the invention, and can reduce the size of the exposure apparatus.

In addition, the instant configuration does not require use of an actuator having a large surface area and emits a large amount of gas, and can increase the degree of vacuum in the vacuum chamber 20. Moreover, this configuration eliminates the exchange operation of the aperture stop, etc., improving the working efficiency of the exposure apparatus.

While this embodiment describes use of the integrator having plural cylindrical surface mirrors, the mirror shape is variable in accordance with the configuration of the illumination optical system, like arc-shape type (or lepidic) corresponding to the illumination area as disclosed in Japanese Patent Applications, Publication Nos. 11-312638 and 2000-223415.

### Second Embodiment

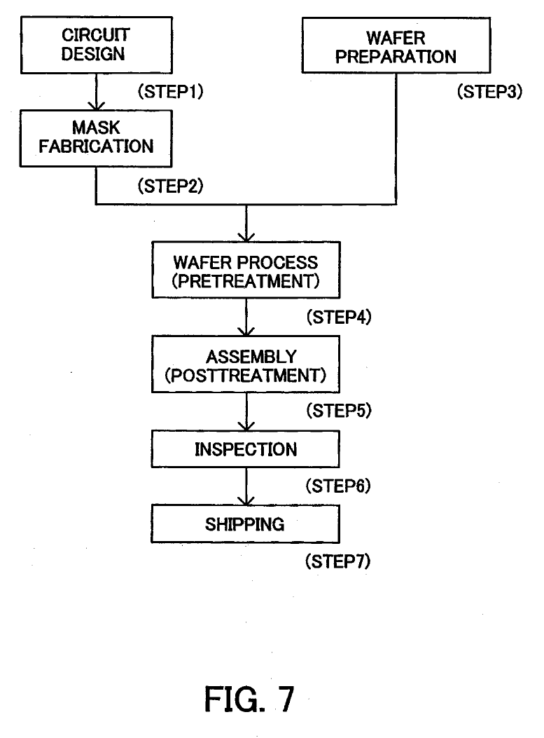

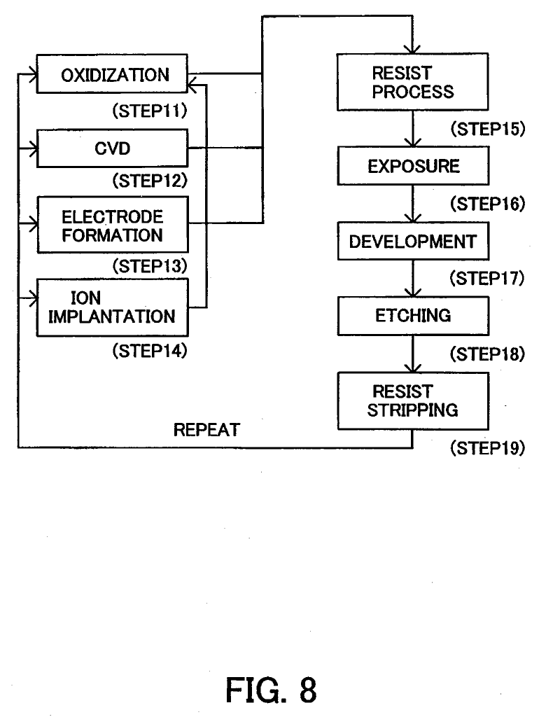

Referring now to FIGS. 7 and 8, a description will be given of an embodiment of a device manufacturing method using the above exposure apparatus 100 according to the first embodiment.

FIG. 7 is a flowchart for explaining how to fabricate devices (i.e., semiconductor chips such as IC and LSI, LCDs, CCDs, and the like). Here, a description will be given of the fabrication of a semiconductor chip as an example.

Step 1 (circuit design) designs a semiconductor device circuit. Step 2 (mask fabrication) forms a mask 14 having a designed circuit pattern. Step 3 (wafer preparation) manufactures a wafer 17 using materials such as silicon.

Step 4 (wafer process), which is also referred to as a pretreatment, forms actual circuitry on the wafer 17 through lithography using the mask 14 and wafer 17.

Step 5 (assembly), which is also referred to as a posttreatment, forms into a semiconductor chip the wafer 17 formed in Step 4 and includes an assembly step (e.g., dicing, bonding), a packaging step (chip sealing), and the like.

Step 6 (inspection) performs various tests for the semiconductor device made in Step 5, such as a validity test and a durability test. Through these steps, a semiconductor device is finished and shipped (Step 7).

FIG. 8 is a detailed flowchart of the wafer process in Step 4. Step 11 (oxidation) oxidizes the wafer 17's surface. Step 12 (CVD) forms an insulating film on the wafer 17's surface. Step 13 (electrode formation) forms electrodes on the wafer 17 by vapor disposition and the like.

Step 14 (ion implantation) implants ions into the wafer 17. Step 15 (resist process) applies a photosensitive material onto the wafer 17. Step 16 (exposure) uses the exposure apparatus 100 to expose a circuit pattern of the mask 14 onto the wafer 17. Step 17 (development) develops the exposed wafer 17. Step 18 (etching) etches parts other than a developed resist image. Step 19 (resist separation) removes disused resist after etching.

These steps are repeated, and multi-layer circuit patterns are formed on the wafer 17. Use of the manufacturing method in this embodiment helps fabricate higher-quality devices than ever.

The device manufacturing method that uses the exposure apparatus 100 and resultant devices constitute one aspect of the present invention.

Further, the present invention is not limited to these preferred embodiments, and various variations and modifications may be made without departing from the scope of the present invention.

This application claims a foreign priority benefit based on Japanese Patent Application No. 2005-031082 filed on Feb. 7, 2005, which is hereby incorporated by reference herein in its entirety as if fully set forth herein.

What is claimed is:

1. An illumination optical system for illuminating a target plane by using light from a light source, said illumination optical system comprising plural displaceable mirrors that are two-dimensionally arranged at specific positions in said illumination optical system.

2. An illumination optical system according to claim 1, wherein each mirror is displaceable in at least one of an inclination and position.

3. An illumination optical system according to claim 1, wherein each mirror has a curved shape.

4. An illumination optical system according to claim 1, wherein the light has a wavelength between 5 nm and 20 nm.

5. An illumination optical system according to claim 1, wherein the specific positions include a pupil position and a position near the pupil position.

6. An illumination optical system according to claim 1, further comprising:
a first optical system for converting the light from the light source into an approximately parallel light;
an integrator that includes the plural mirrors and reflects the light from the first optical system; and
a second optical system for forming an illumination area having a predetermined shape on the target plane by utilizing light from the integrator.

7. An illumination optical system according to claim 1, further comprising a unit for changing an illumination condition to the target plane in accordance with at least one of positions and orientations of the mirrors.

8. An illumination optical system according to claim 1, further comprising:
a measuring unit for measuring a light intensity distribution corresponding to an effective light source distribution of light reflected by the mirrors; and
a controller for controlling displacements of the plural mirrors based on a measurement result by said measuring unit.

9. An exposure apparatus comprising:
an illumination optical system for illuminating an original according to claim 1; and
a projection optical system for projecting an image of a pattern of the original onto a substrate.

10. A device manufacturing method comprising the steps of:
exposing a substrate using an exposure apparatus according to claim 9; and
developing the object that has been exposed.
